(19) United States
(12) Patent Application Publication
(10) Pub. No.: US 2003/0091634 A1
(43) Pub. Date: May 15, 2003

(54) Delayed release tablet of venlafaxin

(76) Inventor: Pawan Seth, Irvine, CA (US)

Correspondence Address: HARNESS, DICKEY & PIERCE, P.L.C., P.O. BOX 828, BLOOMFIELD HILLS, MI, 48303, US

(21) Appl. No.: 10/244,059

(22) Filed: Sep. 13, 2002

Related U.S. Application Data
(63) Continuation-in-part of application No. 09/953,101, filed on Sep. 14, 2001.

(51) Int. Cl. A61K 9/22; A61K 9/36

(52) U.S. Cl. 424/468; 424/480

(57) ABSTRACT

The invention provides a delayed release tablet, comprising: (i) a core comprising an active ingredient and a gelling agent; and (ii) a coating consisting essentially of a water-insoluble, water-permeable film-forming polymer, a plasticizer and a water-soluble polymer.

### CROSS REFERENCE TO RELATED APPLICATIONS

This application is a Continuation-In-Part (CIP) of U.S. Ser. No. 09/953,101, filed Sep. 14, 2001, which is incorporated by reference herein.

### BACKGROUND OF THE INVENTION

There is a need to obtain new release dosage form of venlafaxin, as well as other drugs, especially sustained or delayed.

Formulations for sustained release of medicinal products have various advantages compared to conventional medicinal forms. Firstly, due to the increase in the duration of activity of each dose, the number of doses per day can be reduced, leading to increased comfort for the patient and better compliance to the treatment. Secondly, due to the slowing down of the release of the active principle by the formulation, the maximum plasmatic concentration (Cmax) remains substantially less than that obtained with an identical dose of active principle in a conventional formulation which would release the entire dose in a very short space of time. This decrease in the Cmax allows a reduction in the undesirable effects of the active substance, these effects often being associated with the peaks in concentration. For identical reasons, the sustained-release formulation is preferable in the case of active principles, which have a low therapeutic index, i.e. the ratio between the toxic plasmatic concentration and the therapeutic plasmatic concentration. Reducing the height of the plasmatic peak reduces accordingly the risk of reaching a toxic level. The drawback of controlled-release forms lies in the technology used to manufacture them, which is more complex than for the conventional forms, and in the generally greater cost of the non-active raw materials used. Moreover, the technology commonly used, such as granulation in a mixer or in a fluidized bed, necessitates the use of solvents, such as water or alcohol, which are not always compatible with the chemical stability of the active substance. The object of the invention described in the present patent is to remove all or some of these various drawbacks.

The literature describes many controlled-release pharmaceutical forms, which can be classified into two main groups: osmotic pumps and matrices. Osmotic pumps take advantage of the osmotic pressure exerted by a salt or a hydrophilic polymer in order to release their active principle. Such forms are described, for example, in U.S. Pat. No. 4,327,725 or U.S. Pat. No. 4,612,008. These forms have the drawback of an expensive and delicate manufacturing process. Furthermore, the dissolution profiles obtained with such formulations are often slightly erratic and relatively nonreproducible.

Matricial forms control the release of the active substance through its diffusion across a more or less thick and more or less viscous layer of a gelling substance upon contact with the digestive fluids. By way of example, U.S. Pat. No. 5,840,756 provides the composition of a matricial levodopa tablet and EP-A-0,253,490 itself describes a similar formulation based on levodopa and carbidopa. The advantage of these forms lies in the simple way in which they are manufactured and in the highly reproducible release profiles, which they ensure. On the other hand, the kinetics of release is dependent on the physiochemistry of the active principle (solubility) and of the outside medium (pH, surface tension, etc.).

Thus, there is a need for a sustained-release formulation that would obviate the above-mentioned problems.

### SUMMARY OF THE INVENTION

The invention provides a controlled release tablet comprising:

(i) a core comprising 20 to 70% of active ingredient, 10 to 80% of a gelling agent, and optional conventional excipients; and

(ii) a coating consisting essentially of a water-insoluble, water-permeable film-forming polymer, a plasticizer and a water-soluble polymer.

The invention thus provides a new controlled release composition under the form of a tablet, the core of which comprising the active ingredient and a gelling agent while the coating affords sustained release (the controlled release is obtained thanks to a semi-permeable release coating, free of (monomeric) pore-forming agent). The tablets of the invention exhibit specific dissolution profiles, especially with venlafaxin.

### DETAILED DESCRIPTION OF THE INVENTION

The invention consists in a tablet comprising a core and a coating. The core includes the active ingredient, a gelling agent, and preferably a fusible substance, and optionally conventional excipients, if needed.

The active ingredient is notably venlafaxin or fluoxetine, or a salt thereof as for example venlafaxine or fluoxetine hydrochloride salts, albeit virtually any drug may be used, where the drug is preferably a drug that is unstable in the presence of a liquid such as a solvent—alcohol or water for example—.

The core also comprises a gelling agent which is hydrophilic in nature and which is capable of behaving like a hydrophilic matrix, such as hydroxypropylmethylcellulose, hydroxypropylcellulose, polyethyleneoxide, polyvinylpyrrolidone, xanthan gum, carbomers, carragheen, polyvinyl alcohol or any other substance of the same type known to those skilled in the art. The fusible substance may, for example, be stearic acid, stearyl acid, cetyl alcohol, sorbitan, glyceryl behenate, waxes, polyethylene glycol of molecular weight between 1 500 and 15 000, lanol wax or any other solid substance which is fusible at a temperature of between 35 and 100° C. and which is known to those skilled in the art. Stearic acid is one preferred fusible substance. The above gelling agent, fusible substance, and any other excipient such as a lubricant that may be present can further be found in the relevant literature, for example in the Handbook of Pharmaceutical Excipients. Sodium stearyl fumarate is one preferred lubricant agent.

The relative amounts of ingredients in the core are preferably as follows. The proportion of active ingredient in the core may vary between 10 and 70%, preferably 25 and 60%, of the core dry weight. The proportion of gelling agent in the core may vary between 10 and 80%, preferably 10 and 40%, of the core dry weight. The proportion of fusible substance in the core may vary between 10 and 80%, preferably 20 and 50%, of the core dry weight.

The ingredients are mixed together and the mixture is then granulated by simply heating the inside of the mixer in which the operation is taking place: the melting of the fusible substance then makes it possible to produce a product in granular form without needing to add wetting liquid. The mixer may be heated by any means, such as for example circulating hot water in a jacket, or direct heating with electrical resistance or with microwaves. This technique avoids the traditional granulation techniques using a mixer or a fluidized bed, since these techniques make use of a wetting liquid, which would be detrimental to the stability of the drug. Alternatively, when the fusible substance is not used, any granulation technique may be used, such as in a high shear mixer of a fluidized bed granulator.

The granules are then pressed into tablets. Tablets can be obtained by standard techniques, e.g. on a (rotary) press (for example Manesty Betapress®) fitted with suitable punches. The resulting tablets are hereinafter referred as tablet cores. These tablet cores are then coated with the semi-permeable coating designed to achieve a controlled release of metformin. The coating comprises a water-insoluble, water-permeable film-forming polymer, together with a plasticizer and a water-soluble polymer or substance.

The water-insoluble, water-permeable film-forming polymer can be a cellulose ether, such as ethylcellulose, a cellulose ester, such as cellulose acetate, methacrylic derivatives available from Roehm Pharma under the trade name “Eudragit®”RL, RS and NE, etc. The preferred film-forming polymer is ethylcellulose (available from Dow Chemical under the trade name Ethocel®). The plasticizer can be an ester such as a citrate ester (e.g. triethyl citrate), dibutyl sebacate, dibutyl phthalate, triacetin, an oil such as castor oil, a polyalkyleneglycol such as polyethyleneglycol of various MWs (e.g. between 400 and 6 000), a fatty acid such as stearic acid. The preferred plasticizer are dibutyl sebacate and stearic acid. The water-soluble polymer or substance can be a partially or totally water-soluble hydrophilic substance intended to modulate the film permeability to the outside medium, such as for example polyvinylpyrrolidone, hydroxypropylmethylcellulose, hydrated colloidal silica, sucrose, mannitol or any other substance capable of playing the same role and known to those skilled in the art. It is preferably polyvinylpyrrolidone. Some other excipients can be used in the coating, as for example pigments, etc. The relative amounts of ingredients in the coating are preferably as follows. The proportion of water-insoluble, water-permeable polymer (e.g. ethylcellulose) in the coating may vary between 20 and 85% of the coating dry weight. The proportion of water-soluble polymer or similar substance (e.g. polyvinylpyrrolidone) in the coating may vary between 10 and 75% of the coating dry weight. The proportion of plasticizer (e.g. stearic acid) in the coating may vary between 3 and 40% of the coating dry weight. The relative proportions of ingredients, notably the ratio water-insoluble, water-permeable film-forming polymer to water-soluble polymer or substance and to plasticizer, can be varied depending on the release profile to be obtained (where a more delayed release is generally obtained with a higher amount of water-insoluble, water-permeable film-forming polymer). For example, the following are preferred proportions water-insoluble, water-permeable film-forming polymer/water-soluble polymer/plasticizer: 50-85/10-40/5-20.

The coating process can be as follows. Ethylcellulose, dibutyl sebacate (or stearic acid) and polyvinylpyrrolidone are dissolved in alcoolic solvents such as ethanol or isopropylalcool or a mixture thereof. The resulting solution is sprayed onto the tablet cores, using a coating pan or a perforated turbine or a fluidized bed apparatus. The weight ratio coating/tablet core is comprised e.g. between 1/50 and 5/10, preferably between 2/100 and 20/100. The tablet comprises an amount of active ingredient that can be from 0.5 to 1000 mg, preferably from 5 to 500 mg, per tablet. Surprisingly, it was discovered that the above formulation did provide a controlled (sustained) release though no pore-forming agent was present in the coating.

The invention also provides a venlafaxin controlled release tablet, exhibiting a dissolution profile such that after 2 hours, from 7 to 40% of the venlafaxin is released; after 4 hours, from 15 to 70% of the venlafaxin is released; after 8 hours, from 50 to 100% of the venlafaxin is released; after 12 hours, more than 75% of the venlafaxin is released.

### BEST MODES FOR CARRYING THE INVENTION

A preferred tablet composition comprises:

(i) a core comprised of active ingredient (especially venlafaxin), stearic acid and HPMC; and

(ii) a coating comprised of ethylcellulose, polyvinylpyrrolidone and stearic acid or dibutyl sebacate.

### EXAMPLES

The following examples illustrate the invention without limiting it. The amounts are given per dosage form.

### Example 1

The following formulation is prepared.

| Ingredients | Amount (mg) |
|---|---|
| Venlafaxin | 37.5 |
| Stearic acid | 31.0 |
| HMPC 100,000 cPs | 25.0 |
| Total (dry weight) | 93.5 |

The first two constituents are placed in a mixer of the Kg5 type, Key International, USA, preheated to 65° C. Once the stearic acid is molten, the entire mixture is blended for 2 min at 350 rpm. The product in granular form thus obtained is then passed over an oscillating granulator (ERWEKA TBT), equipped with a screen, which has a mesh size of 1.8 mm. The 100,000 cPs hydroxypropylmethylcellulose is added and the mixture thus obtained is compressed on an “X” rotary tablet press equipped with punches which are 5.45 mm in diameter and have a radius of curvature of 4.53 mm, at a hardness of 65 N.

An active-principle-release assay carried out on these tablets according to the Basket method at 75 rpm with 1 000 ml of purified water gave the following results:

| Time (hour) | 2 | 4 | 8 | 12 |
|---|---|---|---|---|
| Core Example 1 | 65.9 | 89.9 | 100.0 | 100.0 |

The above results are exemplary of an immediate release profile.

These tablet cores are then coated with the following formulation.

| Ingredients | Amount (mg) |
|---|---|
| Tablet cores | 93.5 |
| Ethocel PR100 (ethylcellulose) | 9.14 |
| Kollidon 90F (povidone USP) | 3.91 |
| Stearic acid | 1.30 |
| Total (dry weight) | 107.85 |

Ethocel, povidone and stearic acid are first dissolved in denatured alcohol (105 g). The coating is then carried out in an LDCS Vectoravec perforated turbine with the following operating parameters:

| | | |
|---|---|---|
| Inlet temperature | 38° | C. |
| Exit temperature | 28° | C. |
| Spraying rate | 12 | g/min |
| Atomizing air pressure | 2.0 | bar |
| Rotation rate | 16 | rpm |

An active-principle-release assay carried out on these tablets according to the Basket method at 75 rpm with 1 000 ml of purified water gave the following results:

| Time (hour) | 2 | 4 | 8 | 12 |
|---|---|---|---|---|
| Example 1 | 16.1 | 40.8 | 85.7 | 99.7 |

The above results are exemplary of a delayed release profile.

### Example 2

Example 1 is reproduced, but with the following core formulation:

| Ingredients | Amount (mg) |
|---|---|
| Venlafaxin | 37.5 |
| Stearic acid | 31.0 |
| HMPC 100,000 cPs | 15.0 |
| Fumed silica | 1.5 |
| Total (dry weight) | 79.0 |

The same procedure is followed, the fumed silica being added together with HPMC.

The following coating formulation is used:

| Ingredients | Amount (mg) |
|---|---|
| Tablet cores | 79.0 |
| Ethocel PR100 (ethylcellulose) | 7.50 |
| Kollidon 90F (povidone USP) | 2.95 |
| Stearic acid | 1.55 |
| Total (dry weight) | 91.0 |

The amount of denatured alcohol that is used is this time 150.0 mg; all other parameters are identical.

An active-principle-release assay carried out on these tablets according to the Basket method at 75 rpm with 1 000 ml of purified water gave the following results:

| Time (hour) | 2 | 4 | 18 | 12 |
|---|---|---|---|---|
| Example 2 | 16 | 37.6 | 74.2 | 93.6 |

The above results are exemplary of a delayed release profile.

### Example 3

Example 1 is reproduced, but with the following coating formulation:

| Ingredients | Amount (mg) |
|---|---|
| Tablet cores | 93.5 |
| Ethocel PR100 (ethylcellulose) | 7.50 |
| Kollidon 90F (povidone USP) | 2.95 |
| Dibutyl sebacate | 1.05 |
| Total (dry weight) | 103.5 |

### Example 4

Example 1 is reproduced, but with the following core formulation:

| Ingredients | Amount (mg) |
|---|---|
| Venlafaxin HCl | 42.427 |
| | (corresponds to 37.5 mg of |
| | venlafaxine |
| Glyceryl Behenate | 26.50 |
| Silicon dioxide | 4.00 |
| HMPC 100,000 cP | 19.00 |
| Sodium Stearyl fumarate | 1.00 |
| Colloidal silicon dioxide | 0.473 |
| Total (dry weight) | 93.4 |

The same procedure as described in example 1 is followed; the first three constituents are placed in the mixer in order to obtain granules. Then the Sodium Stearyl fumarate and the Colloidal silicon dioxide are added together with HPMC before the compressing step.

The following coating formulation is used:

| Ingredients | Amount (mg) |
|---|---|
| Tablet cores | 93.4 |
| Ethylcellulose 100 cp | 5.80 |
| Povidone K90 | 2.60 |
| Dibutyl sebacate | 2.10 |
| Total (dry weight) | 103.9 |

In this example the three ingredients are dissolved in a mixture of ethyl alcohol (101.00 mg) and isopropyl alcohol (6.20 mg). All other parameters are identical.

An active-principle-release assay carried out on these tablets according to the Basket method at 75 rpm with 1 000 ml of purified water gave the following results:

| Time (hour) | 2 | 4 | 8 | 12 |
|---|---|---|---|---|
| Example 4 | 20.3 | 41.7 | 69.9 | 83 |

The above results are exemplary of a delayed release profile.

### Example 5

The following core formulation without fusible agent is prepared:

| Ingredients | Amount (mg) |
|---|---|
| Venlafaxin HCL | 42.43 |
| | (corresponds to 37.5 mg |
| | of venlafaxine |
| Xanthan Gum | 17.50 |
| HMPC 3mPa s | 12.50 |
| Povidone K29/32 | 4.50 |
| Sodium Stearyl fumarate | 1.57 |
| Total (dry weight) | 78.5 |

The first three ingredients are mixed and dryed together. The mixture thus obtained is granulated with a solution of the povidone in isopropylic alcool (7.08 mg) which is evaporated during the process. The granules and the sodium stearyl fumarate are blended and dried together before the compression of the mixture.

The following coating formulation is used:

| Ingredients | Amount (mg) |
|---|---|
| Tablet cores | 78.5 |
| Ethylcellulose 100 mPa s | 7.82 |
| Povidone K90 | 3.85 |
| Dibutyl sebacate | 2.33 |
| Total (dry weight) | 92.5 |

Ethylcellulose, Povidone and Dibutyl sebacate are dissolved in a mixture of ethyl alcool (134.48 mg) and isopropyl alcohol (7.08 mg). The coating is then carried as in example 1.

An active-principle-release assay carried out on these tablets according to the Basket method at 75 rpm with 1 000 ml of purified water gave the following results:

| Time (hour) | 2 | 4 | 8 | 12 |
|---|---|---|---|---|
| Example 5 | 11.50 | 33.20 | 71.70 | 82.90 |

The above results are exemplary of a delayed release profile.

The invention is not limited to the specific embodiments described above but can be varied within broad limits by the skilled man.

What is claimed is:

1. A delayed release tablet comprising:
(i) a core comprising 10 to 70% of active ingredient, 10 to 80% of a gelling agent, and optional conventional excipients; and
(ii) a coating consisting essentially by weight, based on the coating weight, of 20 to 85% of a water-insoluble, water-permeable film-forming polymer, of 10 to 75% of a water-soluble polymer or substance and 3 to 40% of a plasticizer.

2. The tablet of claim 1, where the water-insoluble, water-permeable film-forming polymer is ethylcellulose.

3. The tablet of claim 1, where the water-soluble polymer or substance is polyvinylpyrrolidone.

4. The tablet of claim 1, where the plasticizer is stearic acid or dibutyl sebacate.

5. The tablet of claim 1, where the water-insoluble, water-permeable film-forming polymer is ethylcellulose, the water-soluble polymer or substance is polyvinylpyrrolidone and the plasticizer is stearic acid or dibutyl sebacate.

6. The tablet of claim 1, where the weight proportions water-insoluble, water-permeable film-forming polymer/water-soluble polymer or substance/plasticizer are 50-85/10-40/5-20.

7. The tablet of claim 1, where the water-insoluble, water-permeable film-forming polymer is ethylcellulose, the water-soluble polymer or substance is polyvinylpyrrolidone and the plasticizer is stearic acid or dibutyl sebacate, and where the weight proportions water-insoluble, water-permeable film-forming polymer/ water-soluble polymer or substance/plasticizer are 50-85/10-40/5-20.

8. The tablet of claim 1, where the gelling agent is a mixture of at least two gelling agents.

9. The tablet of claim 1, where the gelling agent of the core is hydroxypropylmethylcellulose, xanthan gum, polyvinylpyrrolidone or a mixture thereof.

10. The tablet of claim 1, where the core further comprises a fusible substance, in an amount of 10 to 80%, of the core dry weight.

11. The tablet of claim 10, where the fusible substance is stearic acid or glyceryl behenate.

12. The tablet of claim 1, where the core comprises the active ingredient, hydroxypropylmethylcellulose and stearic acid or glyceryl behenate.

13. A delayed release tablet comprising:
(i) a core comprising 10 to 70% of active ingredient, 10 to 80% of a gelling agent, 10 to 80% of a fusible substance, and optional conventional excipients; and
(ii) a coating consisting essentially by weight, based on the coating weight, of 20 to 85% of a water-insoluble, water-permeable film-forming polymer, of 10 to 75% of a water-soluble polymer or substance and 3 to 40% of a plasticizer.

14. The tablet of claim 13, where the weight proportions active ingredient/gelling agent/fusible substance are 25-60/10-40/20-50.

15. The tablet of claim 13, where the gelling agent is hydroxypropylmethylcellulose and the fusible substance is stearic acid or glyceryl behenate.

16. The tablet of claim 13, where the weight proportions water-insoluble, water-permeable film-forming polymer/ water-soluble polymer or substance/plasticizer are 50-85/10-40/5-20.

17. The tablet of claim 13, where the water-insoluble, water-permeable film-forming polymer is ethylcellulose, the water-soluble polymer is polyvinylpyrrolidone and the plasticizer is stearic acid or dibutyl sebacate.

18. The tablet of claim 1, where the active ingredient is venlafaxin or venlafaxine hydrochloride salt.

19. The tablet of claim 13, where the active ingredient is venlafaxin or venlafaxine hydrochloride salt.

20. A delayed release tablet comprising:
(i) a core comprising 25 to 60% of venlafaxin or venlafaxine hydrochloride salt, 10 to 40% of a gelling agent, 20 to 50% of a fusible substance, and optional conventional excipients; and
(ii) a coating consisting essentially by weight, based on the coating weight, of 50 to 85% of a water-insoluble, water-permeable film-forming polymer, of 10 to 40% of a water-soluble polymer or substance and 5 to 20% of a plasticizer.

21. The tablet of claim 20, where the gelling agent is hydroxypropylmethylcellulose and the fusible substance is stearic acid or glyceryl behenate.

22. The tablet of claim 20, where the water-insoluble, water-permeable film-forming polymer is ethylcellulose, the water-soluble polymer is polyvinylpyrrolidone and the plasticizer is stearic acid or dibutyl sebacate.

23. A delayed release tablet comprising:
(i) a core comprising 10 to 70% of venlafaxin or venlafaxine hydrochloride salt, 10 to 80% of a gelling agent, and optionally conventional excipients; and
(ii) a coating consisting essentially by weight, based on the coating weight, of 40 to 75% of a water-insoluble, water-permeable film-forming polymer, of 10 to 40% of a water-soluble polymer or substance and 10 to 40% of a plasticizer.

24. The tablet of claim 23, where the gelling agent is a mixture of at least two gelling agents.

25. The tablet of claim 23, where the gelling agent of the core is hydroxypropylmethylcellulose, xanthan gum, polyvinylpyrrolidone or a mixture thereof.

26. The tablet of claim 23, where the water-insoluble, water-permeable film-forming polymer is ethylcellulose, the water-soluble polymer is polyvinylpyrrolidone and the plasticizer is stearic acid or dibutyl sebacate.

27. The tablet of claim 23, where the conventional excipient is a lubricant.

28. The tablet of claim 18, exhibiting a dissolution profile such that after 2 hours, from 7 to 40% of the venlafaxin is released; after 4 hours, from 15 to 70% of the venlafaxin is released; after 8 hours, from 50 to 100% of the venlafaxin is released; after 12 hours, more than 75% of the venlafaxin is released.

29. The tablet of claim 19, exhibiting a dissolution profile such that after 2 hours, from 7 to 40% of the venlafaxin is released; after 4 hours, from 15 to 70% of the venlafaxin is released; after 8 hours, from 50 to 100% of the venlafaxin is released; after 12 hours, more than 75% of the venlafaxin is released.

30. The tablet of claim 20, exhibiting a dissolution profile such that after 2 hours, from 7 to 40% of the venlafaxin is released; after 4 hours, from 15 to 70% of the venlafaxin is released; after 8 hours, from 50 to 100% of the venlafaxin is released; after 12 hours, more than 75% of the venlafaxin is released.

31. The tablet of claim 23, exhibiting a dissolution profile such that after 2 hours, from 7 to 40% of the venlafaxin is released; after 4 hours, from 15 to 70% of the venlafaxin is released; after 8 hours, from 50 to 100% of the venlafaxin is released; after 12 hours, more than 75% of the venlafaxin is released.

32. A process for preparing a tablet, said tablet comprising:
(i) a core comprising 10 to 70% of active ingredient, 10 to 80% of a gelling agent, 10 to 80% of a fusible substance, and optional conventional excipients; and
(ii) a coating consisting essentially by weight, based on the coating weight, of 20 to 85% of a water-insoluble, water-permeable film-forming polymer, of 10 to 75% of a water-soluble polymer or substance and 3 to 40% of a plasticizer;
where the process comprises the steps of:
(i) mixing the active ingredient and the fusible substance;
(ii) heating the obtained mixture so that the fusible substance is fused;
(iii) adding and mixing the gelling agent and the optional conventional excipients, whereby granules are obtained;
(iv) compressing said granules into tablet cores; and
(v) coating said tablet cores.

33. The process of claim 32, in which step (v) comprises the sub-steps of:
(a) dissolving the water-insoluble, water-permeable film-forming polymer, the water-soluble polymer or substance and the plasticizer into a solvent; and
(b) spraying the thus-obtained solution onto the tablet cores.

34. A process for preparing a tablet, said tablet comprising:
(i) a core comprising 10 to 70% of active ingredient, 10 to 80% of at least two gelling agents, and optionally conventional excipients; and
(ii) a coating consisting essentially by weight, based on the coating weight, of 40 to 75% of a water-insoluble, water-permeable film-forming polymer, of 10 to 40% of a water-soluble polymer or substance and 10 to 40% of a plasticizer;
where the process comprises the steps of:
i) mixing and drying the active ingredient with a first gelling agent of said at least two gelling agents,
ii) granulating the obtained mixture with a solution of a second gelling agent of said at least two gelling agents,
iii) compressing said granules into tablet cores optionally with conventional excipients; and
iv) coating said tablet cores.

35. The process of claim 34 in which the solution in step ii) is an alcoholic solution of the second gelling agent.

36. The process of claim 34, in which step (iv) comprises the sub-steps of:
(a) dissolving the water-insoluble, water-permeable film-forming polymer, the water-soluble polymer or substance and the plasticizer into a solvent; and
(b) spraying the thus-obtained solution onto the tablet cores.
